(19) United States
(12) Patent Application Publication
(10) Pub. No.: US 2016/0279156 A1
(43) Pub. Date: Sep. 29, 2016

(54) COMPOSITION FOR PREVENTING OR TREATING COLON CANCER, CONTAINING 3,6-ANHYDROL-GALACTOSE

(71) Applicant: KOREA UNIVERSITY RESEARCH AND BUSINESS FOUNDATION, Seoul (KR)

(72) Inventors: Nam Joo Kang, Daegu (KR); Kyoung Heon Kim, Seoul (KR); In-Geol Choi, Seoul (KR); Young Ah Kim, Daegu (KR); Eun-Ju Yun, Seoul (KR); Sae Young Lee, Gyeonggi-do (KR)

(73) Assignee: Korea University Research and Business Foundation, Seoul (KR)

(21) Appl. No.: 14/776,826

(22) PCT Filed: Mar. 11, 2014
(86) PCT No.: PCT/KR14/01991

§ 371 Date: Sep. 15, 2015

(30) Foreign Application Priority Data
Mar. 15, 2013 (KR) 10-2013-0028091

(51) Int. Cl. A61K 31/7004

(52) U.S. Cl. CPC A61K 31/7004

(57) ABSTRACT

The present invention relates to a composition for preventing or treating colon cancer, containing 3,6-anhydro-L-galactose, and more specifically, provides excellent anti-cancer effects by inducing apoptosis of colon cancer cells and inhibiting the growth of colon cancer cells by using 3,6-anhydro-L-galactose separated from red algae-derived agar and purified.

### BACKGROUND

1. Field of the Invention

The present invention relates to a composition for preventing or treating colon cancer which contains 3,6-anhydro-L-galactose and prevents, improves, or treats the colon cancer through the abilities of inducing apoptosis of colon cancer cells and inhibiting the colon cancer cell proliferation of 3,6-anhydro-L-galactose.

2. Discussion of Related Art

Colon cancer is the main cause of cancer-related deaths, and about 102,900 of people have been diagnosed as colon cancer in the USA, and 51,370 of people are estimated to die due to colon cancer. In recent years, westernized eating habits resulted in an increase in colon cancer rates in Asia, and as colon cancer-related factors, consumption of an excess of animal fat, sugar and alcohol, a deficiency of fiber, antioxidant vitamins, vegetables and fruits in the diet or the like are known as main causes of colon cancer. In particular, the excess consumption of animal fat and meat leads to a small amount of feces and a long time for which contents pass through colon to be excreted, in contrast to the consumption of fiber foods such as vegetables, cereals, etc. Moreover, the excretion of bile acids and sterols increases, causing a change in the composition of the bacterial species in colon and an increase in types of bacteria which chemically change these substances. Accordingly, a large amount of carcinogens are generated, the carcinogens remain in the colon for a long time and lengthen contacting time with the colon, and thus colon cancer occurs frequently.

Colon cancer is a representative of solid cancer cells in which a large number of cells grow in an agglomerated form around blood vessels, and is one of the cancers which are difficult to completely cure due to extremely limited treatment options. That is, since drug permeation to the center of agglomerated colon cancer cells is insufficient, colon cancer cells are difficult to be fully removed. Currently, there is almost no method of treating colon cancer with drugs, and only a surgical treatment such as surgery, radiation therapy or the like is a limited method of treating colon cancer, but the colon cancer is hard to cure completely by these treatment methods. Accordingly, many researchers have developed methods by which the growth of colon cancer cells is effectively suppressed, and have conducted many studies to effectively inhibit the growth of the most cancer cells as well as colon cancer cells.

Apoptosis is the important mode of action which exhibits the effect of inhibiting the proliferation of cancer cells by most anticancer drugs, and is active death resulting from the expression and activity of various genes and proteins which are controlled according to the programmed signal in cells. Apoptosis may be easily found in many normal physiological phenomena of living things. For example, apoptosis plays an important role in many processes of morphological change and functional self-organization processes of an immune or nervous system which is found in the initial developmental stages of living things. Further, even after living things are grown up, apoptosis necessarily functions to control the number of tissue homeostasis cells and to remove damaged cells, and also serves as a defense mechanism against infection. Apoptosis is deeply involved in pathogenesis of many diseases. The occurrence of abnormal apoptosis may cause neurodegenerative disease, the immune system abnormality, cardiovascular diseases or the like, and abnormal suppression of apoptosis may be the cause of cancer. Specific examples of diseases in which apoptosis is out of the normal control process and expressed or suppressed abnormally include cancers derived from abnormal expression of the gene such as p53, p16, Bcl-2 or the like, infectious diseases such as HIV, Herpes, and the influenza virus, and autoimmune disorders such as diabetes, rheumatoid arthritis, multiple sclerosis, amyotonia, etc. As such, apoptosis not only plays an important role to normally maintain various physiological functions of living things, but also is closely related to pathogenesis of many diseases.

Apoptosis of each cell constituting an individual is a method for removing abnormal cells from an individual to prevent the development of tumors attributable to improper differentiation induced by differentiation stimulants or genetically damaged cells, that is, a general method for removing genetically-damaged and unrecoverable cells from an individual. This concept is supported by the fact that generally used anti-cancer drugs induce the death of cancer cells through the apoptosis process related to the inhibition of cancer cell proliferation. Accordingly, the disturbance to the apoptosis process induces the survival and growth of cells which are damaged or start to be damaged, and thus the inhibition of apoptosis plays an important role in the carcinogenesis process. Further, materials which are effective for cancer prevention induce the apoptosis of the above-described abnormal cells, and it has been reported that apoptosis induced by these materials is at least related to the cancer preventive activity thereof.

Therefore, the present inventors have found out that 3,6-anhydro-L-galactose separated and purified from the red algae-derived agar induces the apoptosis of cancer cells and exhibits the excellent effect of inhibiting cancer cell proliferation, thereby accomplishing the present invention.

### SUMMARY OF THE INVENTION

Therefore, the present invention is directed to use of 3,6-anhydro-L-galactose to prevent, improve, or treat colon cancer.

In order to achieve the objective, the present invention provides a pharmaceutical composition for preventing or treating colon cancer, the composition includes 3,6-anhydro-L-galactose represented by the following Formula 1:

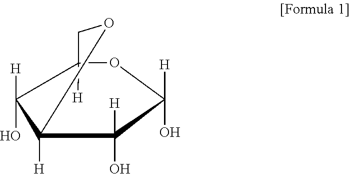 [SMILES: OC1[C@@H](O)OC2CO[C@@H]1[C@@H]2O]

Further, the present invention provides a food composition for preventing or improving colon cancer, the food composition includes 3,6-anhydro-L-galactose of Formula 1.

Further, the present invention provides 3,6-anhydro-L-galactose of Formula 1 used for treating colon cancer.

### BRIEF DESCRIPTION OF THE DRAWINGS

FIG. 1 illustrates an effect of 3,6-anhydro-L-galactose of inhibiting a colon cancer cell proliferation.

FIG. 2 illustrates an effect of inhibiting a tumor transformation, in which an image shows colony formation of colon cancer cells after being subjected to L-AHG according to concentration which is observed with the naked eye, and a graph shows the number of colony formed as described above.

FIG. 3 illustrates an effect of generating apoptosis of colon cancer cells of 3,6-anhydro-L-galactose, in which the number of cells in sub-G1 phase, G0/G1 phase, S phase and G2/M phase using flow cytometry.

FIG. 4 illustrates a result of analyzing DAPI showing the effect of 3,6-anhydro-L-galactose of generating apoptosis.

FIG. 5 illustrates a result of western blotting showing an effect of 3,6-anhydro-L-galactose on expression of apoptosis-related proteins, procaspase-3, procaspase-9, caspase-3, caspase-9, PARP, cleaved PARP, Bcl-2, Bcl-xL and Bax.

FIG. 6 illustrates the result of western blotting showing the effect of 3,6-anhydro-L-galactose on the expression of apoptosis-related proteins, p-p38, p38, p-JNK, JNK and p53.

### DETAILED DESCRIPTION OF EXEMPLARY EMBODIMENTS

Hereinafter, the configuration of the present invention will be described in detail.

The present invention relates to a pharmaceutical composition for preventing or treating colon cancer, the composition includes 3,6-anhydro-L-galactose represented by the following Formula 1:

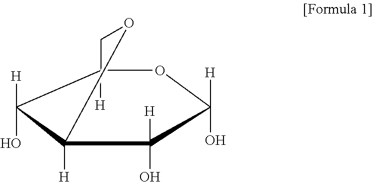 [SMILES: OC1[C@@H](O)OC2CO[C@@H]1[C@@H]2O]

Further, the present invention provides 3,6-anhydro-L-galactose of Formula 1 used for treating colon cancer.

The present inventors took notice of 3,6-anhydro-L-galactose (L-AHG) forming red algae-derived agar in the process of investigating biologically active substances having an anti-cancer effect.

According to one embodiment of the present invention, as compared to a control group not treated with 3,6-anhydro-L-galactose, a group with colon cancer cells treated with 3,6-anhydro-L-galactose show the fact that the colon cancer cell proliferation were inhibited in a concentration-dependent manner, the tumor transformation was inhibited in a concentration-dependent manner, and especially, an excellent effect of inhibiting colony formation was shown at the concentration of 100 μg/mL. Further, it was determined that cells in sub-G1 phase of cell cycle were increased, and especially, a clear apoptosis generation was induced at a high concentration of 100 μg/mL, apoptosis bodies typically shown in apoptosis were formed in colon cancer cells treated with 3,6-anhydro-L-galactose. Further, the expression of caspase-3 and caspase-9 (active form) which are enzymes playing an important role on apoptosis increased and the expression of procaspase-3 and procaspase-9 which are precursors decreased. It was determined that 3,6-anhydro-L-galactose increased the degradation of PARP which is a DNA damage repair-related protein, increased the expression of Bax which is an apoptosis-inducing protein, and inhibited the expression of Bcl-2 and Bcl-xL which are anti-apoptotic proteins. Further, it was determined that, when treated with 3,6-anhydro-L-galactose, the activities of p38 and JNK playing an important role in apoptosis increased and the expression of p53 protein increased.

As described above, a cell cycle is stopped due to the treatment with 3,6-anhydro-L-galactose, the cell proliferation is inhibited and apoptosis occurs, and thus 3,6-anhydro-L-galactose may prevent, improve, or treat colon cancer by inducing apoptosis of colon cancer cells and inhibiting the cancer cell proliferation.

Accordingly, the 3,6-anhydro-L-galactose may be used in a pharmaceutical composition for preventing or treating colon cancer.

The 3,6-anhydro-L-galactose is a monosaccharide forming agar which is a representative carbohydrate moiety forming red algal biomass, may be prepared by a chemical synthesis, or may be prepared using a method in which agarooligosaccharides are prepared from a pretreated agarose, the agarooligosaccharides are decomposed into 3,6-anhydro-L-galactose through an enzymatic saccharification process by agarase and neoagarobiose hydrolase, the 3,6-anhydro-L-galactose thus obtained through the enzymatic saccharification process may be separated and purified using silica gel chromatography and Bio-Gel P2 chromatography which are adsorption chromatography and size exclusion chromatography, respectively, but the present invention is not particularly limited thereto.

According to one embodiment in which the 3,6-anhydro-L-galactose is separated and purified through the enzymatic saccharification process, the pretreatment may be hydrolysis of an agarose using a weak acid, but the present invention is not particularly limited thereto.

Examples of the weak acid include one or two and more of acetic acid, formic acid, succinic acid, citric acid, malic acid, maleic acid, oxalic acid, etc.

Preferably, a weak acid having a concentration in the range of 0.5 to 60% (w/v) may be used in consideration of manufacturing cost and separation of salts generated after weak-acid neutralization. More specifically, a weak acid having a concentration in the range of 20 to 40% (w/v) may be used.

The reaction of the agarose and weak acid may be performed at a temperature in the range of 40 to 150° C. and 100 to 200 rpm for 30 minutes to 6 hours. When the reaction is performed in the above-described range, excessive hydrolysate of the agarose resulting from a weak acid may be minimized.

The agarooligosaccharides generated after the pretreatment react with an exo-type agarase generating a disaccharide to form a neoagarobiose, and is further reacted with neoagarobiose hydrolase to generate D-galactose and 3,6-anhydro-L-galactose which are monosaccharides.

The enzymatic reaction may be performed at a temperature in the range of 20 to 40° C. and 0 to 200 rpm for 30 minutes to 7 days.

More specifically, the enzymatic reaction is as follows.

First, an example of agarase generating neoagarobiose which is a disaccharide by degrading agarooligosaccharides may include an enzyme (hereinafter, referred to as “Aga50D”) which breaks β-1,4-glycosidic bonds between D-galactose and 3,6-anhydro-L-galactose of agarose.

The agarase may be separated and purified from a supernatant or a supernatant liquid of cultures of Saccharophagus degradans, and may be generated and separated by other strains than Saccharophagus degradans or an artificial chemical synthesis method using a genetic engineering recombinant technology.

The reaction of the agarooligosaccharides and agarase may be performed at a temperature in the range of 20 to 40° C. and 0 to 200 rpm for 30 minutes to 7 days. More specifically, the reaction may be performed at a temperature in the range of 25 to 35° C. and 100 to 150 rpm for 1 to 4 days.

Neoagarobiose generated by the enzymatic reaction is decomposed into D-galactose and 3,6-anhydro-L-galactose by neoagarobiose hydrolase.

The neoagarobiose hydrolase may be separated and purified from a supernatant or a supernatant liquid of cultures of Saccharophagus degradans, and may be generated and separated by other strains than Saccharophagus degradans or an artificial chemical synthesis method using a genetic engineering recombinant technology.

The reaction of the neoagarobiose and neoagarobiose hydrolase may be performed at a temperature in the range of 20 to 40° C. and 0 to 200 rpm for 30 minutes to 7 days. More specifically, the reaction may be performed at a temperature in the range of 25 to 35° C. and 100 to 150 rpm for 1 to 4 days.

Silica gel chromatography and Bio-Gel P2 chromatography which are adsorption chromatography and size exclusion chromatography, respectively, are sequentially performed on the degradation product of the neoagarobiose, and thereby 3,6-anhydro-L-galactose with high purity of about 96% may be separated and purified.

The pharmaceutical composition of the present invention may further include a pharmaceutically acceptable carrier.

The pharmaceutically acceptable carrier includes a carrier and a vehicle which are generally used in a medical field, and more specifically, includes ion exchange resins, alumina, aluminum stearate, lecithin, serum proteins (e.g., human serum albumin), buffer substances (e.g., various phosphates, glycine, sorbic acid, potassium sorbate, a partial glyceride mixture of saturated vegetable fatty acid), water, salts or electrolytes (e.g., protamine sulfate, disodium hydrogen phosphate, potassium hydrogen phosphate, sodium chloride and zinc salt), colloidal silica, magnesium trisilicate, polyvinyl pyrrolidone, cellulose-based substrates, polyethylene glycol, sodium carboxymethylcellulose, polyarylates, waxes, polyethylene glycol, wool fat, or the like, but the present invention is not limited thereto.

Further, the pharmaceutical composition according to the embodiment of the present invention may further include lubricants, wetting agents, emulsifiers, suspensions, preservatives, or the like in addition to the above-described compositions.

According to an aspect of the present invention, the pharmaceutical composition according to the present invention may be prepared in the form of an aqueous solution for a parenteral administration, and preferably, a buffering solution, such as Hank's solution, Ringer's solution, or physically buffered saline, may be used. A substrate which may increase the viscosity of a suspension, such as sodium carboxymethyl cellulose, sorbitol, or dextran, may be added to an aqueous injection suspension.

The composition according to the present invention may be administrated through a route of systemic administration or topical administration, and may be formulated into suitable dosage forms for these administrations. For example, upon oral administration, the pharmaceutical composition may be mixed with an inactive diluent or edible carrier, sealed in a hard or soft gelatin capsule or pressed into tablets for administration. For the oral administration, the active ingredient may be mixed with an excipient to be used in form of ingestible tablet, buccal tablet, troche, capsule, elixirs, suspension, syrup, wafer, etc.

Various dosage forms such as dosage forms for parenteral administration or the like may be prepared according to techniques known in the related field or a commonly used technique. 3,6-anhydro-L-galactose is dissolved well in a saline or a buffer solution, and thus is stored in a freeze-dried state, and then the effective dose of 3,6-anhydro-L-galactose may be formulated into a solution, in a suitable dosage form for intravenous, subcutaneous, intramuscula, intraperitoneal, transdermal administration or the like immediately prior to administration to a saline or a buffer solution.

The dosage of the pharmaceutical composition according to the present invention may vary depending on various factors, including dosage form, the patient's age, weight, gender, state of health, diet, the time of administration, the route of administration, excretion rate, sensitivity, etc. For example, the pharmaceutical composition according to the embodiment of the present invention may be administered in a single dose or in multiple doses per day at a daily dose in the range of 0.1 to 1, 000 mg/kg, and preferably, at a daily dose in the range of 10 to 100 mg/kg for adults.

Further, the present invention relates to a food composition for preventing or improving colon cancer including 3,6-anhydro-L-galactose of Formula 1.

The food composition according to the present invention may be added to food ingredients to provide a food such as drinks, teas, spices, chewing gum and confectionery, or to provide capsule, powder or suspension, and may have specific beneficial effects in terms of health when ingested.

The food composition according to the present invention may be ingested daily, and thus is very useful because the food composition may have an excellent effect of preventing or improving colon cancer.

When the 3,6-anhydro-L-galactose is used for food additives, the 3,6-anhydro-L-galactose may be added as it is or may be used with other foods or food components, and may be used according to a general method. A mixing amount of effective components may be suitably determined according to the purpose thereof (for prevention, health, or therapeutic treatment). Generally, in the manufacture of foods or beverages, the food composition according to the present invention is added at an amount of 15 wt % or less, and preferably, at an amount of 10 wt % or less based on the total weight of a raw material. More specifically, the food composition according to the embodiment of the present invention is added at 0.0001 to 10 wt %. Even more specifically, the food composition according to the embodiment of the present invention is added at 0.001 to 1 wt %. However, when ingested for a long time for the purpose of health and hygiene or health control, the amount of the food composition may be below the above-described range. Further, since there is no problem in safety, the effective components may be used at an amount above the above-described range.

The type of the food is not particularly limited. Examples of foods to which the above-described substance may be added include meats, sausages, breads, chocolates, candies, snacks, confectioneries, pizzas, Ramen, noodles, gums, dairy products including ice creams, various types of soups, beverages, teas, drinks, alcoholic beverages, vitamin complexes, etc.

When the food composition according to the present invention is used as a health drink, the health drink may include various flavor agents, natural carbohydrates or the like as an additional component like general drinks Examples of the natural carbohydrates include monosaccharides such as glucose and fructose; disaccharides such as maltose and sucrose; polysaccharides such as dextrin and cyclodextrin, and sugar alcohols such as xylitol, sorbitol, and erythritol. Examples of flavoring agents include natural flavoring agents such as thaumatin and stevia extracts, synthetic flavoring agents such as saccharin and aspartame, etc. The natural carbohydrate may be included in an amount of about 0.01 to 0.04 g, preferably about 0.02 to 0.03 g per 100 mL of the health drink, that is, the food composition according to the present invention.

In addition, the food composition according to the present invention may contain various nutrients, vitamins, electrolytes, flavoring agents, colorants, pectic acid and its salt, alginic acid and its salt, organic acids, protective colloidal thickeners, pH adjusting agents, stabilizers, preservatives, glycerin, alcohols, carbonizing agent used in carbonated drinks, etc.

Moreover, the food composition according to the present invention may contain the flesh of fruits for the manufacture of natural fruit juices, fruit juice beverages, and vegetable beverages. These compositions may be used independently or mixed. The content of the additive is not particularly important, but may be generally selected within the range of 0.01 to 0.1 parts by weights per 100 parts by weight of the food composition according to the present invention.

Advantages and features of the present invention, and method for achieving thereof will be apparent with reference to the accompanying figures and detailed description that follows. However, it should be understood that the present invention is not limited to the following embodiments and may be embodied in different ways, and that the embodiments are given to provide complete disclosure of the invention and to provide thorough understanding of the invention to those skilled in the art, and the scope of the invention is limited only by the accompanying claims and equivalents thereof. Like components will be denoted by like reference numerals throughout the specification.

### EXAMPLE 1

### Measurement of Effect of Inhibiting Colon Cancer Cell Proliferation of 3,6-anhydro-L-galactose

MTT assay was performed to measure cell viability by 3,6-anhydro-L-galactos. HCT-116 cells (Korean cell line bank, Korea) which are human derived colon cancer cells were plated on a 96-well plate, with 100 μl per each well at an concentration of 3×10⁴ cells/mL, and then were stabilized for 4 hours in a CO₂ incubator at 37° C. for 4 hours. 3,6-anhydro-L-galactose was prepared according to the concentration and 100 μl of 3,6-anhydro-L-galactose was added to each incubated cell, and cells were incubated for 3 days under the same conditions. Here, 20 μl of a reagent of 3-(4,5-dimethyl-thiazol-2-yl)-2,5-diphenyl-tetrazolium bromide (MTT) prepared at a concentration of 5 mg/mL was added to each incubated cell, the incubated cells were incubated under conditions of 37° C. and 5% CO₂ in a dark for 2 hours, and the formation of formazan was observed. The MTT reagent was removed, 200 μl of DMSO was added to dissolve formazan generated as above, and then absorbance was measured at a wavelength of 570 nm using Microplate (ELISA) reader. The cell vitality of a control group treated with nothing was defined as 100%, and the cell vitality of a group treated with 3,6-anhydro-L-galactose was expressed as a percentage based on the control group.

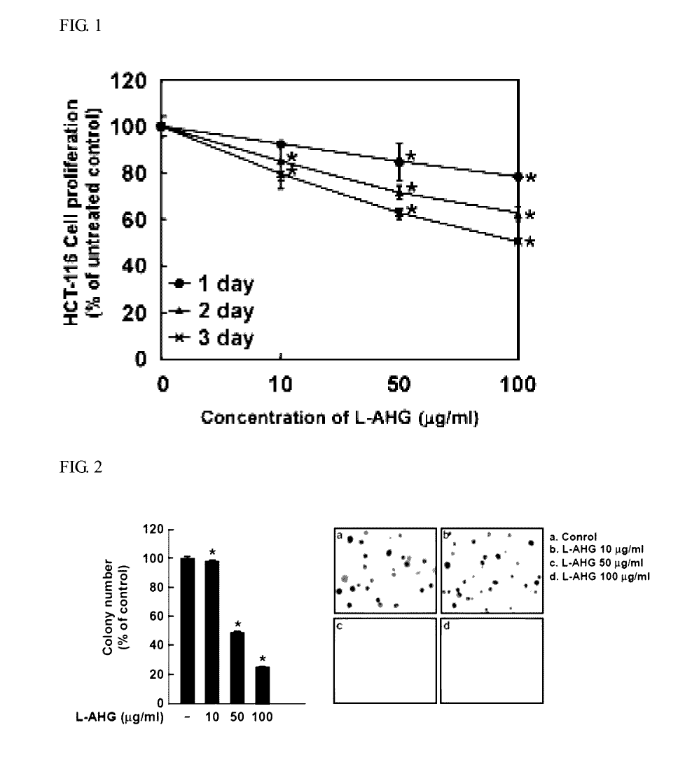

As shown in FIG. 1, the proliferation of colon cancer cells treated with 3,6-anhydro-L-galactose was inhibited in a concentration-dependent manner as compared to the control group not treated with 3,6-anhydro-L-galactose.

### EXAMPLE 2

### Measurement of Effect of Inhibiting Tumor Transformation of 3,6-anhydro-L-galactose

Soft agar assay was performed to measure the effect of inhibiting a tumor transformation of 3,6-anhydro-L-galactose.

In order to evaluate the influence of 3,6-anhydro-L-galactose on the formation of colony of cancer cells, HCT-116 cells (Korean cell line bank, Korea) which are human derived colon cancer cells were seeded with L-AHG at a concentration of 10, 50, 100 μg/mL on a 6-well plate, and were incubated for two weeks at 37° C.

The picture showing the degree of colony formation was taken using microscope after two weeks, and the number of colonies was counted. The number of colonies was calculated according to the following expression.

As shown in FIG. 2, 3,6-anhydro-L-galactose inhibited the tumor transformation of cancer cells in a concentration-dependent manner, and especially, exhibited an excellent effect of inhibiting the colony formation at a concentration of 100 μg/mL.

### EXAMPLE 3

### Measurement of Effect of Inducing Apoptosis of 3,6-anhydro-L-galactose

In order to quantitatively determine the degree of inducing apoptosis of cancer cells by 3,6-anhydro-L-galactose, the experiment was performed as below through flow cytometry using propidium iodide (PI, Sigma-Aldrich Corporation, MO, USA) staining with respect to cells in sub-G1 phase of cell cycle which shows the degree of apoptosis.

Specifically, HCT-116 cells (Korean cell line bank, Korea) which are human derived colon cancer cells were plated on a 60 mm dish, and were incubated under conditions of 37° C. and 5% CO₂ for 24 hours using an RPMI 1640 medium to which FBS is added. A medium was removed after 24 hours and was replaced with a medium containing 3,6-anhydro-L-galactose at a concentration of 25, 50 and 100 μg/mL, and cells were incubated under the same conditions until about 80% or more of the bottom of the well is filled with the cells.

After incubation for 4 days, DNAs of cancer cells were stained using PI. To this end, the cells attached to the bottom of the well were collected using trypsin, and were centrifuged to remove a supernatant, were cleaned using a cold phosphate buffer solution (PBS) once, 1 mL of 70%-absolute ethanol was added thereto, and the solution was fixed at −20° C. for 24 hours. Anhydrous ethanol was removed after centrifugation, cells were cleaned using a PBS twice, were resuspended in 1 mL of a PBS containing 20 μg/mL-RNase and 200 μg/mL-propidium iodide, were reacted at 37° C. in a dark for 10 minutes, and were analyzed through flow cytometry. Data obtained from 20,000 cells with respect to each data file was collected.

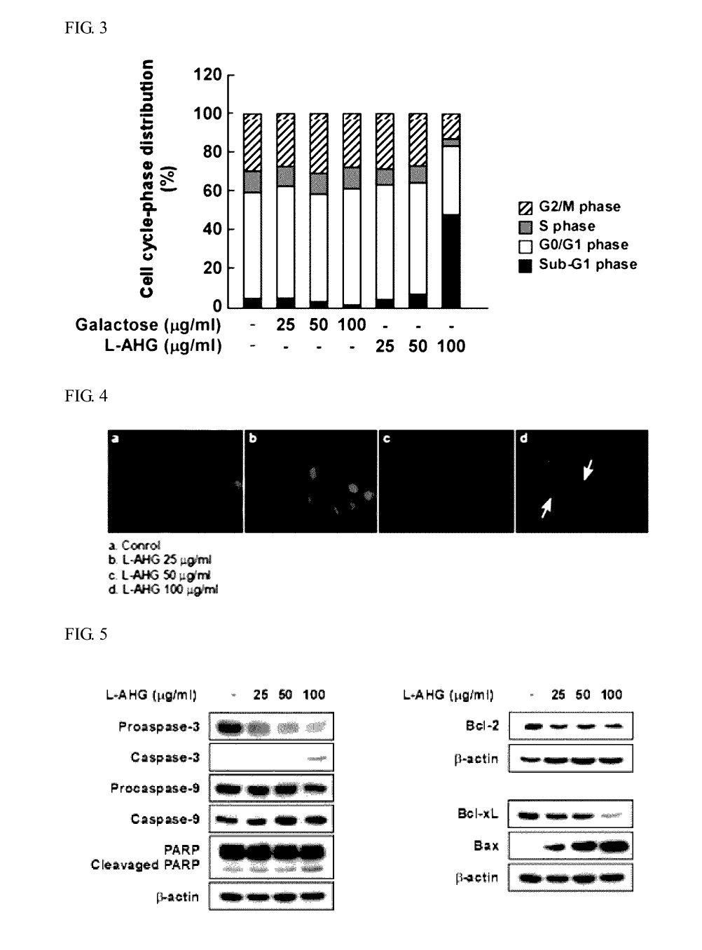

As shown in FIG. 3, 3,6-anhydro-L-galactose increased sub-G1 phase-cells of cell cycle, and especially, induced clear apoptosis at a high concentration of 100 μg/mL. From this result, it was determined that 3,6-anhydro-L-galactose arrests the progress of cell cycle, and induces apoptosis.

Further, as shown in the result of analyzing DAPI of FIG. 4, it was determined that apoptosis bodies typically shown in apoptosis were formed in colon cancer cells treated with 3,6-anhydro-L-galactose.

### EXAMPLE 4

### Measurement of Expression of Apoptosis-Related Proteins Due to 3,6-anhydro-L-galactose

In order to observe the mechanism of inducing apoptosis of HCT-116 cells by 3,6-anhydro-L-galactose determined from the result of the example, a western blotting was performed to determine the change in expression of genes which play an important role in control of inducing apoptosis.

Bcl-2 is an oncogene protein, but is not involved in the cell proliferation in contrast to other oncogene proteins, and has a performance of controlling cell survival, that is, a performance of inhibiting apoptosis. Bax is a protein which belongs to Bcl-2 family, and has a performance of promoting apoptosis.

In order to measure the expression of apoptosis-related proteins of 3,6-anhydro-L-galactose, HCT-116 cells (Korean cell line bank, Korea) which are derived from human colon cancer cells were seeded on a 60 mm dish, and were incubated under conditions of 37° C. and 5% CO₂ for 24 hours using an RPMI 1640 medium supplemented with FBS . The medium was removed after 24 hours and was replaced with a medium containing 3,6-anhydro-L-galactose at a concentration of 25, 50 and 100 μg/mL, and cells were incubated under the same conditions until about 80% or more of the bottom of the well is filled with the cells.

Western blotting was performed after incubation for 5 days. To this end, cells were cleaned with a PBS twice, cells attached to the bottom of the well were collected using a lysis buffer, and the solution was centrifuged at 14,000 rpm for 10 minutes. After each supernatant was obtained, the protein concentration of the supernatant of each cell was quantified using bovine serum albumin (BSA) as a reference material according to the method using a protein assay kit (Bio-Rad Laboratories, USA).

Each protein thus prepared (20 to 40 μg) was degenerated and separated using 10% gel sodium dodecyl sulphate (SDS)-polyacrylamide gel electrophoresis (PAGE), and was transferred to a PVDF membrane at 100 mA for 2 hours. Thereafter, proteins were immersed in a TBS-T solution containing 5%-skim milk to react for 2 hours such that non-specific proteins were blocked, and were cleaned using TBS-T once. Here, rabbit polyclonal anti-human caspase-3 Ab (Cell Signaling Technology, Inc., USA) was used as an antibody to determine the expresson amount of procaspase-3 and caspase-3 of each cell, rabbit polyclonal anti-human caspase-9 Ab (Cell Signaling Technology, Inc., USA) was used as an antibody to determine the expression amount of procaspase-9 and caspase-9 of each cell, rabbit polyclonal anti-human PARP Ab (Cell Signaling Technology, Inc., USA) was used as an antibody to determine the expression amount of PARP and cleaved PARP, rabbit polyclonal anti-human Bcl-2 Ab (Cell Signaling Technology, Inc., USA) was used as an antibody to determine the expression amount of Bcl-2, rabbit polyclonal anti-human Bcl-xL Ab (Cell Signaling Technology, Inc., USA) was used as an antibody to determine the expression amount of Bcl-xL, rabbit polyclonal anti-human Bax Ab (Cell Signaling Technology, Inc., USA) was used as an antibody to determine the expression amount of Bax, mouse monoclonal anti-human phospho-p38 Ab (Becton, Dickinson and Company, USA) was used as an antibody to determine the expression amount of p-p38, mouse monoclonal anti-p38 Ab (Santa Cruz Biotechnology, Inc., USA) was used as an antibody to determine the expression amount of p38, rabbit polyclonal anti-human phospho-SAPK/JNK Ab (Cell Signaling Technology, Inc., USA) was used as an antibody to determine the expression amount of p-JNK, rabbit polyclonal anti-human JNK (Santa Cruz Biotechnology, Inc., USA) was used as an antibody to determine the expression amount of JNK, and rabbit monoclonal anti-human p53 Ab (Cell Signaling Technology, Inc., USA) was used as an antibody to determine the expression amount of p53. The above-described antibodies were used by diluting in TBS-T at the ratio of 1:1000, and the reaction was performed at 4° C. for overnight. Anti-rabbit IgG (Santa Cruz Biotechnology, Inc., USA) and anti-mouse IgG (Santa Cruz Biotechnology, Inc., USA) to which HRP is bonded were used as a secondary antibody by diluting at the ratio of 1:5000, and the reaction was performed at room temperature for 2 hours. Thereafter, proteins were cleaned using TBS-T 4 times, were reacted with an ECL substrate (Amersham™, UK) for 1 to 3 minutes, and were sensitized to an X-ray film to analyze the change in expression of apoptosis-related proteins of each cell.

As shown in FIG. 5, the expression of caspase-3 and caspase-9 (active form) which are enzymes playing an important role in apoptosis increased, and the expression of procaspase-3 and procaspase-9 which are precursors thereof decreased. It was determined that 3,6-anhydro-L-galactose increased the degradation of PARP which is a DNA damage repair-related protein, increased the expression of Bax which is an apoptosis-inducing protein, and inhibited the expression of Bcl-2 and Bcl-xL which are anti-apoptotic proteins.

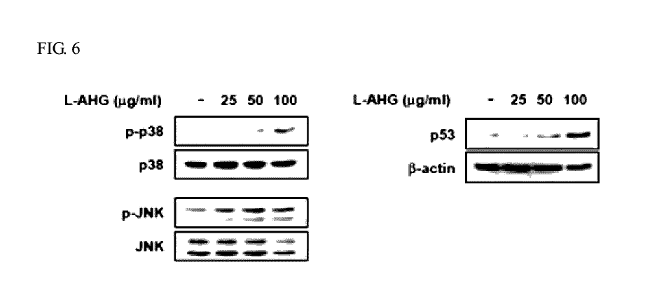

Further, as shown in FIG. 6, it was determined, the activities of p38 and JNK playing an important role in apoptosis increased and the expression of p53 proteins increased when treated with 3,6-anhydro-L-galactose.

Accordingly, it was determined that a cell cycle was stopped, and thereby the cell proliferation was inhibited and apoptosis occurred.

3,6-anhydro-L-galactose according to the present invention may be used as a therapeutic agent for colon cancer.

In the present invention, 3,6-anhydro-L-galactose exhibits excellent anti-cancer activity by inhibiting the growth of colon cancer cells and inducing apoptosis of colon cancer cells, and thus may be effectively used for preventing, treating, or improving colon cancer.

1. A method for preventing or treating colon cancer, comprising;
administering to a subject in need thereof, a pharmaceutical composition comprising a therapeutically effective amount of 3,6-anhydro-L-galactose represented by the following Formula 1:

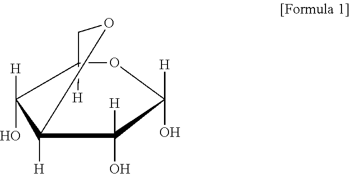 [SMILES: OC1[C@@H](O)OC2CO[C@@H]1[C@@H]2O]

2. The method of claim 1, wherein 3,6-anhydro-L-galactose is separated and purified from red algae-derived agar.

3. The method of claim 2, wherein 3,6-anhydro-L-galactose is separated and purified through a process in which agarooligosaccharides are prepared from a pretreated agarose and an enzymatic saccharification process progresses between the agarooligosaccharides, and agarase and neoagarobiose hydrolase.

4. The method of claim 1, 3,6-anhydro-L-galactose is combined with a pharmaceutically acceptable carrier.

5. A method for preventing or improving colon cancer, comprising;
administering to a subject in need thereof, a food composition comprising an effective amount of 3,6-anhydro-L-galactose represented by the following Formula 1:

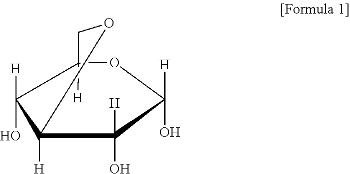 [SMILES: OC1[C@@H](O)OC2CO[C@@H]1[C@@H]2O]

6. The method of claim 5, wherein 3,6-anhydro-L-galactose is separated and purified from red algae-derived agar.

7. The method of claim 6, wherein 3,6-anhydro-L-galactose is separated and purified through a process in which agarooligosaccharides are prepared from a pretreated agarose and an enzymatic saccharification process processes between the agarooligosaccharides, and agarase and neoagarobiose hydrolase.

8. (canceled)
